(19) United States
(12) Patent Application Publication
(10) Pub. No.: US 2023/0416174 A1
(43) Pub. Date: Dec. 28, 2023

(54) POLYMER SEPARATION MEMBRANE FOR PURIFYING METHANE

(71) Applicant: Axiom Angewandte Prozesstechnik Ges.m.b.H, Ebreichsdorf (AT)

(72) Inventor: Aleksander MAKARUK, Wien (AT)

(21) Appl. No.: 18/033,203

(22) PCT Filed: Oct. 21, 2021
(86) PCT No.: PCT/EP2021/079207

§ 371 Date: Apr. 21, 2023

(30) Foreign Application Priority Data
Oct. 22, 2020 (AT) A60315/2020
Feb. 15, 2021 (EP) 21157181.5

(51) Int. Cl. C07C 7/144; B01D 71/40; B01D 53/22; B01D 53/26; B01D 69/02

(52) U.S. Cl. CPC C07C 7/144; B01D 71/40; B01D 53/228; B01D 53/26; B01D 69/02; B01D 2311/10; B01D 2257/108; B01D 2257/80; B01D 2256/245; B01D 2325/32; B01D 2257/104

(57) ABSTRACT

The use of polymer separation membranes to selectively separate CO₂ and H₂ from CH₄ in a membrane separation step for purifying methane contained in an optionally pre-dried product gas mixture of a methanation method which contains CH₄, H₂ and CO₂ is described.
a) The separation is carried out at an operation temperature T_{B} between −20° C. and 100° C.; and
b) the polymer membranes
   b1) are able to simultaneously separate CO₂ and H₂ from CH₄,
   b2) have a higher selectivity for the separation of CO₂ than of H₂ from CH₄, i.e., a ratio α1/α2<1, and
   b3) have a glass transition temperature T_{g} that is lower than the operation temperature T_{B}.

The present invention relates to the use of polymer separation membranes for purifying methane obtained by methanation.

### STATE OF THE ART

Methanation, i.e., the production of methane—also referred to as “synthetic natural gas” (SNG)—through hydration of carbon monoxide and dioxide, has been steadily gaining importance in recent years because energy supply has increasingly shifted towards renewable energy sources due to climate change and dwindling fossil fuel resources. In particular, methanation using CO₂ from the atmosphere, which was regarded as inefficient and not implementable on an industrial scale due to the low CO₂ content of air (approx. 400 ppm) and the high energy demands of chemical separation methods until recently, has been increasingly becoming the focus of attention of process engineers. In the meantime, hydrogen, which is a necessary reaction partner, has increasingly been produced in a sustainable manner through water electrolysis with electricity produced by wind and solar energy (CH₄ obtained through methanation also being referred to as “wind gas” or “solar gas”), so that the field is seeing the continuous development of improved methods.

One main focus has been the use of methane obtained through methanation as synthetic fuel for so-called “natural gas vehicles.” Electric and hybrid vehicles are still strongly on the rise, however, experts do not consider electric drives to be the technology of the future. The reason is that a production of such vehicles in significantly larger scales would entail the danger of raw material shortages, e.g., of lithium and cobalt as well as rare earths, and in addition storage capacity, working life and cycle stability of drive batteries are still relatively limited.

In methanation methods based on carbon dioxide from the atmosphere for producing methane for use as synthetic fuel, however, requirements regarding the purity of both the CO₂ separated from the air and the methane obtained in the process have to be set high. For example, when methane is fed into the natural gas grid, the limits for the concentrations of H₂ and CO₂ therein are in the single-digit percentage range, e.g., according to OEVGW guideline G31 a maximum of 4 vol % of H₂ and 2 vol % of CO₂. Recently, however, efforts have been made to adjust these limits by lowering the limit for CO₂, which causes corrosion effects, even further, e.g., below 1 vol % or even below 0.5 vol %, and increasing that for H₂, which increases the calorific value of the natural gas, e.g., to 10 vol %.

However, the methanation of carbon dioxide according to the general reaction equation

4H₂+CO₂→CH₄+2H₂O

is never completed under technical conditions so that the product gas may comprise, in addition to CH₄ and H₂O, substantial amounts of unreacted starting products H₂ and CO₂. These are not only undesirable in most applications of the obtained methane, but may also be recycled for methanation. For the latter reason, usually excessive H₂ is used in the catalytic methanation of CO₂ (contained in the ambient air) in order to increase conversion of CO₂ to CH₄, whereafter excessive hydrogen is recycled.

In addition, higher hydrocarbons are usually formed as side products during the methanation reaction, in particular those having two to four carbon atoms, which, however, are not undesirable components because they increase the specific calorific value of the gas mixture and behave similar to methane during membrane separation and can be purified together therewith.

In addition to cryo, adsorption (e.g., pressure swing adsorption) and absorption methods, membrane separation methods are often used for separating methane from other side and unreacted starting products. For example, the Vienna University of Technology disclosed in WO 2015/017875 A1 a method for storing energy in which H₂ and CO₂ are produced separately by water electrolysis, from which CH₄ is subsequently formed by methanation, which can then be fed into a natural gas grid. For purifying the product gas from methanation, a membrane separation system using gas separation membranes is disclosed, which are able to selectively separate CO₂ and H₂ and optionally also H₂O from the CH₄ produced, with polymerfilm, metal and ceramic membranes being disclosed as suitable. Preferably, however, gas separation occurs in one single membrane separation step, i.e., by means of membranes having higher selectivity for all three gases to be removed than for CH₄. For this purpose, membranes made of plastic, in particular polyimide membranes are disclosed, for which a selectivity of 60 for the H₂/CH₄ separation and of 20 for CH₂/CH₄ is disclosed. In addition, a selectivity of more than 100 up to 1000 is disclosed for H₂O/CH₄ separation.

Here, selectivity is given as parameter a, which is the so-called ideal selectivity for a gas pair, i.e., the relation of the permeabilities P of the two gas components for a particular membrane type. For the purposes of the present invention, in the following these are referred to as α1, α2, and α3, respectively, according to the following formulas:

$$a 1 = \frac{{P}_{H 2}}{{P}_{CH 4}} a 2 = \frac{{P}_{CO 2}}{{P}_{CH 4}} a 3 = \frac{{P}_{H 20}}{{P}_{CH 4}}$$

In this connection, Tanihara et al., J. Membr. Sci, 160, 179-186 (1999), disclose, for polyimides produced from biphenyl-tetracarboxylic acid dianhydride (BPDA) and various aromatic diamines, selectivities of α1=130 and α2=40 for H₂/CH₄ and CO₂/CH₄ separations at 50° C., while Yang et al., Polymer 42, 2021-2029 (2001), disclose values of α1=80 and α2=44 (at 35° C.) for a polyimide produced from 4,4′-(hexafluoroisopropylidene)diphthalic anhydride (6FDA) and 2,6-dimethyl-3,7-diaminodibenzothiophene 5,5-dioxide (DBBT).

In addition to polyimide membranes, other plastic membranes known for purifying methanation product gases due to their similarly high selectivities for H₂/CH₄ and CO₂/CH₄ separations are, for example, polysulfone and cellulose acetate membranes, which allow very efficient gas membrane separation because they guarantee very high gas yields and purities as well as relatively low recompression efforts.

Herein, recompression effort means the energy input required for applying the pressure present during methanation to the permeate, recycled to the reactor and CH₄ depleted, of gas membrane separation. This pressure is usually several up to several dozens of bars, occasionally even 100 bar or more, to shift the equilibrium of the methanation reaction according to the principle of Le Chatelier and Braun towards the product side because the gas volume decreases during the reaction (five molecules educt become three molecules product).

Here, selectivity of the H₂/CH₄ separation using membranes according to the state of the art, such as polyimide, polysulfone, and cellulose acetate membranes, is consistently higher than with CO₂/CH₄, i.e., α1>α2 and α1/α2>1, respectively, which also decreases H₂ consumption, which is used in excess and then recycled. As has been cited from WO 2015/017875 A1 before, the selectivity of a H₂O/CH₄ separation, i.e., the value of α3, is generally highest.

All plastic membrane materials mentioned have in common that they have to be present in a glassy or energy-elastic, brittle state at the respective operation temperature of membrane separation in order to achieve the high selectivities of gas separation. Therefore, preferred membrane materials are those having high proportions of aromatic rings, in particular bulky aromatics, in the polymer chains in order to provide high glass transition temperatures T_{g}. This avoids that the membranes have to be cooled during separation operation in order to maintain the polymers in their glassy state below the glass transition temperature. However, such plastics, including in particular polyimides comprising relatively rigid polymer chains, are hardly or not meltable and insoluble in most organic solvents, which makes their processing complicated and expensive.

In addition, due to the limit values for the concentrations of H₂ and CO₂ in a methane flow to be fed into the natural gas grid, which are considerably higher for H₂ than for CO₂, it is disadvantageous that the above plastic membranes used for membrane separation consistently show higher selectivity for the separation of hydrogen than of carbon dioxide from methane. For this reason, product gas flows of methanation methods have to be subjected to a higher number of membrane separation steps or cycles in order to reduce the CO₂ content of the methane to an admissible value. This means that, during continuous operation with recycling of the permeate from the membrane separation enriched with CO₂ and H₂, larger amounts of permeate have to be recycled and recompressed, which considerably increases energy consumption and reduces the cost-effectiveness of the system.

A further group of plastic membranes that are often used for gas separation are so-called elastomer membranes, which are, contrary to the polyimide membranes described above, used above their glass transition temperatures T_{g}, i.e., in their rubbery state. These mostly consist of polyethers, such as poly(tetramethylene glycol) or polytetrahydrofuran (PolyTHF), poly(ethylene glycol) (PEG), and poly(propylene glycol) (PPG), or also polyether-block-polyamide (PEBA) copolymers. Normally, they have—sometimes considerably—higher selectivity for CO₂ than for H₂, which is why they lend themselves to the use in the separation of CO₂ from exhausts.

For example, Li et al., J. Membr. Sci. 369, 49-58 (2011), disclose permeability experiments with membranes made of PEG, PPG, PolyTHF membranes available on the market under the trade name Terathane®, as well as composite membranes made of combinations of these plastics. Initially, the permeability of the membranes for six different gases, namely O₂, N₂, H₂, He, CH₄, and CO₂, were examined at different pressures, from which their selectivities for the separation of CO₂ from binary gas mixtures were calculated. The values (i.e., α1 values) obtained for the separation of CO₂/CH₄ were approximately 7 and those for the separation of CO₂/H₂ were below 5. Application fields mentioned for such membranes are the separation of CO₂/H₂ from synthetic gas, CO₂/N₂ from the air, CO₂/CH₄ for natural gas purification, and CO₂/O₂ in food packaging.

Another working group disclosed, in several publications, investigations regarding the permeability and selectivity of PEBA (available under the trade name Pebax®) and PEBA/PEG composite membranes, first testing only pure gases (Car et al., J. Membr. Sci. 307, 88-95 (2008)), but later also gas mixtures (Car et al., Sep. Purif. Technol. 62, 110-117 (2008)). Here, the separation of CO₂/CH₄ showed α1 mean values of 15 and CO₂/H₂ of 10. The latter article discloses a possible use of these membranes for separating CO₂ from exhaust gases, such as from continuous-flow heaters, coal burning power plants and oil refineries.

Finally, Ahmadpour et al., J. Nat. Gas Sci. Eng. 21, 518-523 (2014), disclose using a PEBA membrane as well as a PEBA/PVC composite membrane for purifying natural gas and measuring the permeabilities of these membranes for pure CO₂ and CH₄ under varying pressures and temperatures, from which subsequently the selectivities α1 were again calculated. The values obtained were between 22 and 35, with those of the composite membrane with PVC being hardly any better than the values for PEBA alone. The permeability of the composite membrane for hydrogen was not determined, however, it is to be assumed that it will not differ much from that of the PEBA membrane.

Against this background, it was an object of the invention to develop a new method for producing methane by reducing CO₂ with H₂ followed by a membrane separation of the product gas that at least partly overcomes the above disadvantages.

### DISCLOSURE OF THE INVENTION

The present invention achieves this object by providing the use of polymer separation membranes being able to selective separate CO₂ and H₂ from CH₄ in a membrane separation step for purifying methane contained in an optionally pre-dried product gas mixture of a methanation method, which comprises CH₄, H₂ and CO₂, the use according to the present invention being characterized in that
a) separation is carried out at an operation temperature T_{B} between −20° C. and 100° C.; and
b) the polymer membranes
   b1) are able to simultaneously separate CO₂ and H₂ from CH₄,
   b2) have higher selectivity for the separation of CO₂ than of H₂ from CH₄, i.e., a ratio α1/α2<1, and
   b3) have a glass transition temperature T_{g} that is lower than the operation temperature T_{B}.

In other words, a method for producing methane is provided herein, which comprises the following steps:
a methanation step in which, by reducing CO₂ with H₂, a product gas is formed that comprises H₂O, H₂ and CO₂ in addition to CH₄;
optionally a drying step in which H₂O is removed from the product gas; and
a membrane separation step for purifying the methane, wherein the gas mixture obtained by drying and containing CH₄, H₂ and CO₂ is subjected to separation using separation membranes being able to selectively separate CO₂ and H₂ from CH₄; and which is characterized by
a) the separation in the membrane separation step being conducted at an operation temperature T_{B}, between −20° C. and 100° C.; and
b) using polymer membranes that
   b1) are able to simultaneously separate CO₂ and H₂ from CH₄,
   b2) have higher selectivity for the separation of CO₂ than of H₂ from CH₄, i.e., a ratio α1/α2<1, and
   b3) have a glass transition temperature T_{g} that is lower than the operation temperature T_{B}.

By using polymer membranes for purifying a methanation product gas, which are, in diametrical contrast to the state of the art, able to separate CO₂ from CH₄ with higher selectivity than H₂, i.e. with α2>α1 or α1/α2<1, and which are used above their glass transition temperatures, i.e. in their rubbery states, it is possible to provide methane having limit values for the concentration of CO₂ and H₂ suitable for being fed into a natural gas grid in a simple and cost-effective manner by methanation of CO₂ and subsequent membrane purification. Due to the inverted selectivity ratio between α1 and α2, a lower number of membrane separation steps or cycles or also smaller membrane surfaces are sufficient to bring the CO₂ concentration below the prescribed limit value. And the inventive use of membranes above their glass transition temperatures also allows for higher temperatures during separation, which can, in some cases, increase separation efficiency.

The reason why the use of membranes with such selectivity ratios for purifying methanation product gases is completely unknown in the state of the art is mainly that their selectivities for separating the respective gas, CO₂ or H₂, from CH₄ are considerably lower than those of plastic, in particular polyimide membranes that are usually used for this purpose. For example, the plastic membrane having the highest selectivity for CO₂ and H₂ among the tested inventive membranes has a value of only 35 for α2 (CO₂/CH₄) and of only 2.5 for α1 (H₂/CH₄), while for polyimide membranes, as cited above, values for α1 (H₂/CH₄) of sometimes well above 100 and for α2 (CO₂/CH₄) of at least 40 are disclosed. This is evidenced by comparative examples, where the inventor even achieved an α2 value of 70 in one experiment.

In addition, as mentioned above, methanation product gases often contain much higher concentrations of H₂ than of CO₂, especially when excessive H₂ is used in the reaction. However, when using membranes with α1/α2<1 according to the present invention, there is no requirement for an excess, or at least no large excess, of hydrogen because unreacted CO₂ can in any case be more selectively separated from the methane produced that H₂— and, of course, can be recycled, too. This reduces the overall costs of recycling because smaller amounts of gas have to be recycled according to the present invention.

As evidenced by the following examples and comparative examples, the present invention allows the separation of CO₂ and H₂ from CH₄ with a significantly higher energy efficiency than according to the state of the art, even though less separation-efficient membranes are used, which was extremely surprising for the person skilled in the art.

It would be possible to heat the membranes working above their glass transition temperatures in their rubbery states according to the invention to higher temperatures, but this is not necessary—on the contrary: the examples show that with the membranes used in preferred embodiments of the invention, the selectivities α1 and α2 for H₂/CH₄ and CO₂/CH₄ gas separations increase with decreasing temperatures. At the same time, the selectivity ratio α1/α2 surprisingly decreases further when lowering the operation temperature. This means that the selectivity α2 for CO₂/CH₄ separation increases more strongly when approaching the glass transition temperature, i.e., when lowering the rubbery properties of the membranes, than the selectivity α1 of H₂/CH₄ separation.

The material of polymer separation membranes is not particularly limited according to the present invention, as long as it has a glass transition temperature lower than the respective operation temperature, i.e., is in its rubbery state at the operation temperature, and causes the membranes made thereof to be able to simultaneously separate CO₂ and H₂ from CH₄— with a higher selectivity for CO₂/CH₄ separation than for H₂/CH₄ separation according to the invention. A person of average skill in the art can easily determine plastic membranes suitable for this purpose.

Separation membranes preferred according to the invention, which have already been proved their worth, include, for example, those made of polyethers, poly(urethaneurea) elastomers, polyethers, polysiloxanes, and thermoplastic poly(ether-block-polyamide) (PEBA) copolymers, of which PEBA copolymer membranes are particularly preferred because they allow the surprising effects mentioned above to be obtained reproducibly due to their particularly low ratios between α1 and α2.

With regard to feeding the purified methane into natural gas grids, i.e. to current and planned future limit values, preferred embodiments of the invention lower the content of CO₂ in the methane purified in the membrane separation step to below 2 vol %, more preferably below 1 vol %, most preferably below 0.5 vol %; and/or the content of H₂ in the purified methane below 10 vol %, below 8 vol %, below 4 vol %, or below 2 vol %, particularly preferably below 10 vol % or below 8 vol %.

Further preferred embodiments of the present invention are, due to the advantages determined for the membranes used according to the invention, characterized by the gas separation being conducted at an operation temperature T_{B} between 0° C. and 60° C., preferably between 5° C. and 30° C., more preferably between 10° C. and 25° C. In this way, the method is conducted above the glass transition temperature T_{g} of the membrane plastics, while no overly complex temperature control is required for the separation, and in particularly preferred embodiments of the invention operation can even take place at the respective ambient temperatures outdoors—even during cold seasons.

Finally, it is also possible according to the present invention to separate not only CO₂ and H₂, but at the same time H₂O from the methane produced, because the membranes used according to the invention normally show the highest selectivity α3 for the last separation step. In this way, pre-drying of the methanation product gas does not have to be complete or can, in particular situations, even be omitted entirely.

### SHORT DESCRIPTION OF THE DRAWINGS

In the following, the present invention will be described in more detail by means of nonlimiting examples and referring to a single drawing,

FIG. 1, schematically showing the procedure of a method or plant, respectively, for producing methane by methanation according to the state of the art using the inventive membranes during the membrane separation step.

### EXAMPLES

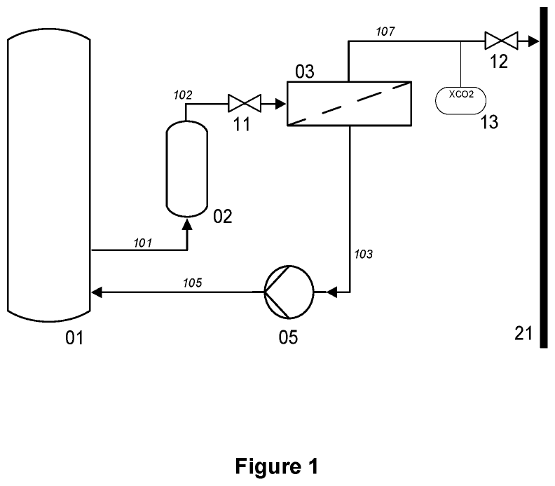

As mentioned above, the method and corresponding plant schematically shown in FIG. 1 correspond to a relatively simple embodiment according to the state of the art. Here, the actual methanation reaction through hydrogenation of carbon dioxide—preferably originating from ambient air—is conducted in reactor 01 according to the reaction equation

4H₂+CO₂→CH₄+2H₂O

resulting in a product gas mixture 101 rich in water and methane (and, as mentioned at the beginning, optionally further hydrocarbons, which will, however, not be discussed in further detail).

At position 02, before gas separation, this mixture is subjected to a pretreatment step normally comprising (pre-)drying as well as an optional temperature adjustment and/or removal of particles and other components (e.g., from the environmental air) potentially detrimental to the membranes such as ammonia or higher hydrocarbons, as well as the application of pressure required for membrane separation to the gas flow. The pre-treated product gas 102 passes through a control valve 11 into the gas membrane separator 03, which comprises at least one membrane separation step using polymer membranes to be used according to the invention and separates the gas mixture into at least one high-pressure retentate flow 107 and at least one low-pressure permeate flow 103.

Due to the higher selectivity of the membranes for the gas components CO₂ and H₂ compared to CH₄, CO₂ and H₂ are simultaneously enriched in the permeate flow 103 and depleted in the retentate flow 107 according to the present invention.

According to the state of the art, separator 02 uses membranes having the highest possible selectivities for H₂ and CO₂ compared to CH₄, i.e., membranes having the highest possible values for α1 and α2, in order to separate the largest possible amount of these two gases from the product gas flow in each separation step. These are all polymer membranes, in particular polyimide membranes, in their glassy states below their glass transition temperatures and they all show a higher selectivity for the separation of H₂ than of CO₂ from CH₄, i.e., a ratio α1/α2>1. However, this is particularly disadvantageous in view of feeding the purified methane into a natural gas grid because a larger number of membrane separation steps or cycles or larger membrane surfaces are required in order to lower the CO₂ content of the methane to the admissible limit value. At the same time, according to the state of the art, the H₂ concentration is decreased to values that are far below the admissible limit values, which unnecessarily increases the recyclate volume flow 103 requiring larger amounts of energy for recompression by a compressor 05.

For this reason, the present invention uses polymer membranes showing higher selectivities for the separation of CO₂ than of H₂ from CH₄, i.e., a ratio α1/α2<1, because the limit values for the CO₂ concentration are, as mentioned at the beginning, often only half of those for H₂. This considerably reduces the number of required membrane separation steps before feeding into the gas grid.

In preferred embodiments, the separator according to the invention still comprises a plurality of membrane separation stages of the polymer membranes to be used according to the invention so that in the retentate flow 107, i.e., in the purified methane,
the content of CO₂ is decreased below 2 vol %, more preferably below 1 vol %, most preferably below 0.5 vol %; and/or
the content of H₂ is decreased below 10 vol %, below 8 vol %, below 4 vol %, or below 2 vol %, particularly preferably below 10 vol % or below 8 vol %;

in particular both, because in this way the purified methane has a sufficiently low concentration of CO₂ and H₂ in the retentate 107 in order to—after the concentration is measured using an analyzer 13—be able to be fed into a natural gas grid shown as bold line 21.

Subsequently, in a compressor 105, the pressure desired for methanation is applied to permeate 103 which is refed into the reactor 01 as compressed recyclate 105.

Due to the lower limit value of CO₂, gas analyzer 13 is preferably mainly a CO₂ analyzer. Based on the concentration measurement values from analyzer 13, the control valve 11, the control valve 12, the compressor 05, and the gas pretreatment 02 can be controlled, if required, to adjust the temperature and/or the pressure. In this way, the ratio of the volume flows of retentate 107 and permeate 103 can also be adjusted.

As mentioned above, the pretreatment step at position 02 may also comprise temperature adjustment in order to adjust the inventive operation temperature T_{B} between −20° C. and 100° C. or to set an operation temperature preferred according to the invention between 0° C. and 60° C., more preferably between 5° C. and 30° C., most preferably between 10° C. and 25° C., limits included, if required. This guarantees that the operation temperature T_{B} is higher than the glass transition temperature T_{g} of the polymer membrane to be used according to the invention when a particular type of membrane is to be used.

Here, the respective selection of the polymer membranes mainly depends on their selectivity ratio α1/α2 and the composition of the product gas mixture produced in the respective reactor 01, i.e., on the concentration of CO₂ and H₂ therein. For example, when excessive hydrogen is used for a catalytic methanation and the H₂ concentration in the product gas flow 101 is (considerably) higher than that of CO₂, the polymer membranes used in separator 03 are, for obtaining suitable H₂ concentrations in the retentate 107, preferably those having a selectivity ratio α1/α2 less far below or even just below 1, i.e., which are able to separate CO₂ and H₂ almost equally well from CH₄. In this way, when a H₂ concentration in retentate 107 of, for example, below 4 vol %, which is admissible for feeding into a natural gas grid according to OEVGW guideline G31, is obtained, very probably the CO₂ concentration also lies below the admissible 2 vol %. However, in other cases, for example when excessive CO₂ is available for methanation, e.g., when obtaining CO₂ from environmental air, the invention preferably uses membranes having the smallest possible selectivity ratio α1/α2 in order to separate considerably more CO₂ than H₂ from the product gas flow in every separation step.

### Example 1, Comparative Example 1

For a theoretical calculation of the energy consumption of a continuous operation of a plant constructed as shown in FIG. 1, it was assumed that a methanation method was conducted through hydrogenation of CO₂ according to the equation

4H₂+CO₂→CH₄+2H₂O

in reactor 01, followed by 100% drying of the product gas 101 in dryer 02 and subsequent purification of the product gas 103 in separator 03 by separating CO₂ and H₂ from CH₄ by means of a respective polyimide membrane commonly used therefor in its glassy state and a polymer membrane according to the invention in its rubbery state, both at ambient temperature. In addition, it was assumed that a pressure of 60 bar is maintained in reactor 01 in order to shift the chemical equilibrium towards the product side, that drying is ideally conducted without pressure loss, and that permeate 103 enriched in CO₂ and H₂ is continuously recycled from separator 03 to reactor 01 after having been brought back to the reaction pressure of 60 bar in compressor 05. For the membranes, the following selectivities α1 and α2 were assumed for the H₂/CH₄ (α1) and CO₂/CH₄ (α2) separations.

### Comparative Example 1

Polyimide membrane (state of the art): α1=70 α2=30 α1/α2=2.33

### Example 1

Polyether-block-polyamide (PEBA) membrane: α1=2 α2=20 α1/α2=0.10

These lie within the common selectivities and selectivity ratios for the respective membrane types, as will be shown by the examples and comparative examples below.

Finally, a maximum admissible CO₂ content in retentate 107 of only 0.5 vol % was assumed, which is well below the limit value according to the OEVGW guideline G31, however, is taken with regard to reductions of this limit value planned for the future, as mentioned above, in order to be allowed to keep feeding the purified methane into the natural gas grid after such a reduction. At the same time, however, the limit value for the H₂ content assumed is above this guideline because it is planned to increase it to up to 10 vol %.

Here, the difference in energy consumption for operation of the method is essentially based on the compression power of compressor 05, which has to compress different permeate volume flows depending on the gas separation membranes used in the separator. The higher the pressure in the reactor, the higher are the compression efforts saved by the present invention.

The values calculated based on the above assumptions are shown in Table 1 overleaf.

TABLE 1

| | | Comparative | |
|---|---|---|---|
| Description | Unit | Example 1 | Example 1 |
| Membrane selectivity α1, H₂/CH₄ | | 70 | 2 |
| Membrane selectivity α2, CO₂/CH₄ | | 30 | 20 |
| CO₂ content in methanation product gas | [vol %] | 2.0 | 2.0 |
| H₂ content in methanation product gas | [vol %] | 8.0 | 8.0 |
| CH₄ content in methanation product gas | [vol %] | 90.0 | 90.0 |
| Methanation product gas overpressure | [bar] | 60.0 | 60.0 |
| Methanation product gas volume flow rate | [Sm³/h] | 6000.0 | 6000.0 |
| CO₂ content in permeate | [vol %] | 9.6 | 13.2 |
| H₂ content in permeate | [vol %] | 44.8 | 13.3 |
| CH₄ content in permeate | [vol %] | 45.6 | 73.5 |
| Permeate overpressure | [bar] | 2.0 | 2.0 |
| Permeate volume flow rate | [Sm³/h] | 993 | 385 |
| CO₂ content in retentate before feeding into grid | [vol %] | 0.5 | 0.5 |
| H₂ content in retentate | [vol %] | 0.7 | 7.3 |
| Retentate volume flow rate | [Sm³/h] | 5007 | 5615 |
| Required compressor power | [kW] | 378 | 265 |
| Improvement of energy efficiency in gas treatment by | [%] | | 30% |

Since the PEBA membrane is only able to separate H₂ and CO₂ less selectively from CH₄ and thus has considerably lower absolute values for α1 and α2 (α1=2, α2=20) compared to the polyimide membrane (α1=70, α2=30), the permeate contains larger amounts of CH₄ (73.5 vol % compared to 45.6 vol %). This is also the main reason why such membranes have so far not been used for the inventive purpose according to the state of the art.

However, the inventive gas membrane separation results in a permeate volume flow of only 385 Sm³/h compared to 993 Sm³/h according to the state of the art, which is why 30% less compressor power is required in order to repressurize the permeate with a pressure of 60 bar. For even higher pressures, energy savings would be correspondingly higher.

### Examples 2 to 7, Comparative Examples 2 to 4

Table 2 overleaf shows several membrane types together with their respective selectivities α1 and α2 and selectivity ratios α1/α2, namely polymer membranes known according to the state of the art to be used for gas membrane separation of a methanation product gas in their glassy state below their glass transition temperatures T_{g} as Comparative Examples 2 to 4 (C2 to C4) as well as polymer membranes to be used according to the invention in their rubbery state above their glass transition temperatures having inverted selectivity ratios as Examples 2 to 7 of the invention (E2 to E7).

Here, the values for α1 and α2 were either taken from relevant literature or determined by the inventor in own experiments. Here, pure gas permeation experiments with the respective gas, i.e. CH₄, CO₂ or H₂, were conducted at room temperature with different feed gas pressures, the linear proportionality factor was calculated from the measurement results as the quotient of the arithmetic mean of the measured flow rates at different pressures and the respective pressure (m²/bar), and the quotient of the proportionality factors for H₂ and CH₄ was taken as α1 and that of the factors for CO₂ and CH₄ was taken as α2 for the respective membrane.

TABLE 2

| | | Temperature | | | | |
|---|---|---|---|---|---|---|
| Example | Membrane material | [° C.] | α1 | α2 | α1/α2 | Source |
| C2 | Polyimide BPDA - arom. diamine | 40 | 130 | 40 | 3.25 | Tanihara et al. ^{c} |
| C3 | Polyimide BPDA - arom. diamine | 25 | 190 | 70 | 2.71 | Experiment |
| C4 | Polyimide 6FDA-DBBT | 35 | 80 | 45 | 1.777 | Yang et al. ^{d} |
| E2 | Terathane ® 2900 (PolyTHF) ^{a} | 35 | 1.5 | 7 | 0.21 | Li et al. ^{e} |
| E3 | Polydimethylsiloxane (PDMS) | 23 | 1.5 | 4 | 0.375 | Experiment |
| E4 | Pebax ® MH 1657 ^{b} | 30 | 2 | 16 | 0.125 | Car et al. ^{f} |
| E5 | Pebax ® MH 1657 ^{b} | 10 | 2.5 | 26 | 0.096 | Car et al. ^{f} |
| E6 | Pebax ® MV 1074 ^{b} | 27 | 2 | 16 | 0.125 | Car et al. ^{f} |
| E7 | PVC/Pebax ® MH 1657 | 20 | 2.5 | 35 | 0.07 | Ahmadpour et al. ^{g} |

^{a} Commercially available membrane made of poly(tetramethyleneglycol) ether (polytetrahydrofuran, PolyTHF)
^{b} Commercially available membranes made of polyether-block-polyamide copolymers (PEBA)
^{c} Tanihara et al., J. Membr. Sci. 160, 179-186 (1999).
^{d} Yang et al., Polymer 42, 2021-2029 (2001).
^{e} Li et al., J. Membr. Sci. 369, 49-58 (2011).
^{f} Car et al., J. Membr. Sci. 307, 88-95 (2008).
^{g} Ahmadpour et al., J. Nat. Gas Sci. Eng. 21, 518-523 (2014).

The results from Table 2 show that the selectivity ratios α1/α2 of the inventive polymer membranes are—contrary to the membranes according to the state of the art in their glassy states—not only below 1 but are typically also an order of magnitude below those of commonly used membranes.

In addition, a comparison of Examples 4 and 5 shows that the selectivity for H₂ and CO₂ with regard to CH₄, i.e., α1 and α2, for membranes used according to the present invention in their rubbery states increase with decreasing temperatures, with α2 increasing more than α1, so that the selectivity ratio α1/α2 decreases further when lowering the operation temperature. Consequently, according to the present invention, a targeted increase of the temperature during gas separation will be unnecessary in most cases.

### Examples 8 and 9, Comparative Examples 5 to 7

A calculation of further examples of the present invention and of comparative examples was based on the operation of a plant analogous to Example 1 and Comparative Example 1, using the selectivities of the commercially available membranes of Comparative Examples 2 to 4 and Examples 5 and 6 listed in Table 2 above.

The results are shown Table 3 overleaf.

TABLE 3

| Description | Unit | Comp. 5 | Comp. 6 | Comp. 7 | Ex. 8 | Ex. 9 |
|---|---|---|---|---|---|---|
| Membrane selectivity α1, H₂/CH₄ | | 130 | 190 | 80 | 2.5 | 2 |
| Membrane selectivity α2, CO₂/CH₄ | | 40 | 70 | 45 | 26 | 16 |
| CO₂ content in methanation product gas | [vol %] | 2.0 | 2.0 | 2.0 | 2.0 | 2.0 |
| H₂ content in methanation product gas | [vol %] | 8.0 | 8.0 | 8.0 | 8.0 | 8.0 |
| CH₄ content in methanation product gas | [vol %] | 90.0 | 90.0 | 90.0 | 90.0 | 90.0 |
| Methanation product gas overpressure | [bar] | 60.0 | 60.0 | 60.0 | 60.0 | 60.0 |
| Methanation product gas volume flow rate | [Sm³/h] | 6000.0 | 6000.0 | 6000.0 | 6000.0 | 6000.0 |
| CO₂ content in permeate | [vol %] | 10.0 | 11.6 | 10.9 | 13.3 | 10.3 |
| H₂ content in permeate | [vol %] | 48.9 | 55.3 | 49.7 | 14.2 | 12.0 |
| CH₄ content in permeate | [vol %] | 41.1 | 33.1 | 39.4 | 72.5 | 77.7 |
| Permeate overpressure | [bar] | 2.0 | 2.0 | 2.0 | 2.0 | 2.0 |
| Permeate volume flow rate | [Sm³/h] | 946 | 812 | 864 | 702 | 915 |
| CO₂ content in retentate before feeding | [vol %] | 0.5 | 0.5 | 0.5 | 0.5 | 0.5 |
| into grid | | | | | | |
| H₂ content in retentate | [vol %] | 0.34 | 0.6 | 1.0 | 7.2 | 7.3 |
| Retentate volume flow rate | [Sm³/h] | 5054 | 5188 | 5136 | 5298 | 5085 |
| Required compressor power | [kW] | 360 | 309 | 329 | 267 | 348 |

The values for the required compressor power of compressor 05 show that the membrane used according to the present invention in Example 8, which—like the one in Example 1—had a selectivity ratio α1/α2 of approximately 1:10, again yielded better results than all commercially available membranes having inverted selectivity ratios regularly used for product gas purification according to the state of the art.

The required compressor power calculated for inventive Example 9 is just above the average of the three comparative examples, however, for identical CO₂ contents, the two inventive examples are able to achieve an H₂ content in the purified methane that is even up to approximately 20 times higher than according to the state of the art, after that of Example 1 was already 10 times higher than that of Comparative Example 1.

In addition, the very high selectivity ratio α1/α2 of approximately 2.7 in Comparative Example 5 was based on laboratory measurement values of the inventor (see Table 2, Comparative Example 3, “Experiment”), which will certainly not be achievable in practice during operation of a gas purification plant, which is why also in this case significantly larger amounts of permeate would have to be recycled and recompressed, which would further increase the required compressor power. Thus, for Comparative Example 6 a realistically required compressor power would lie between those of Comparative Examples 5 and 7—and thus in the range of Example 9.

Examples 10 to 17, Comparative Examples 8 to 15 In the calculation examples overleaf, pair comparisons were made between the membrane of Example 8 according to the invention and the prior art membrane of Comparative Example 7 by varying various process parameters, again assuming a maximum CO₂ content of 0.5 vol % and a maximum H₂ content of 10 vol % in the purified methane.

TABLE 4

| | Membrane | | Methanation product gas | | | | Permeate | | | Retentate | | Compressor | |
|---|---|---|---|---|---|---|---|---|---|---|---|---|---|
| | selectivity | | | | CO₂ | H₂ | | CO₂ | H₂ | CO₂ | H₂ | req. | |
| | H₂/CH₄ | CO₂/CH₄ | Vol. flow | Pressure | content | content | Pressure | content | content | content | content | power | Saved |
| Example | (α1) | (α2) | [Sm³/h] | [bar] | [vol %] | [vol %] | [bar] | [vol %] | [vol %] | [vol %] | [vol %] | [kW] | energy |
| B10 | 2.5 | 26 | 6000.0 | 60.0 | 3.0 | 12.0 | 0.5 | 16.5 | 19.9 | 0.23 | 10.0 | 535 | 11% |
| V8 | 80 | 45 | | | | | | 13.5 | 59.9 | 0.5 | 0.6 | 604 | |
| B11 | 2.5 | 26 | 6000.0 | 30.0 | 3.0 | 12.0 | 0.5 | 15.1 | 19.7 | 0.3 | 10.0 | 419 | 12% |
| V9 | 80 | 45 | | | | | | 12.6 | 55.3 | 0.5 | 0.7 | 475 | |
| B12 | 2.5 | 26 | 6000.0 | 30.0 | 3.0 | 12.0 | 2.0 | 13.1 | 19.4 | 0.48 | 10.0 | 317 | 16% |
| V10 | 80 | 45 | | | | | | 11.0 | 47.5 | 0.5 | 0.9 | 379 | |
| B13 | 2.5 | 26 | 6000.0 | 30.0 | 3.0 | 12.0 | 5.0 | 9.4 | 18.4 | 0.5 | 9.5 | 283 | 10% |
| V11 | 80 | 45 | | | | | | 8.6 | 36.2 | 0.47 | 1.0 | 313 | |
| B14 | 2.5 | 26 | 6000.0 | 30.0 | 4.0 | 12.0 | 5.0 | 11.8 | 17.8 | 0.5 | 9.4 | 312 | 7% |
| V12 | 80 | 45 | | | | | | 10.9 | 34.3 | 0.5 | 0.7 | 336 | |
| B15 | 2.5 | 26 | 6000.0 | 30.0 | 2.0 | 10.0 | 2.0 | 10.0 | 17.1 | 0.5 | 8.7 | 251 | 15% |
| V13 | 80 | 45 | | | | | | 8.5 | 47.4 | 0.5 | 1.4 | 295 | |
| B16 | 2.5 | 26 | 6000.0 | 30.0 | 3.0 | 10.0 | 2.0 | 13.3 | 16.4 | 0.5 | 8.5 | 310 | 12% |
| V14 | 80 | 45 | | | | | | 11.7 | 42.3 | 0.5 | 0.8 | 353 | |
| B17 | 2.5 | 26 | 6000.0 | 30.0 | 3.0 | 10.0 | 2.0 | 15.6 | 16.4 | 1.0 | 9.0 | 218 | 17% |
| V15 | 80 | 45 | | | | | | 13.0 | 49.2 | 1.0 | 2.2 | 265 | |

It can be seen that the same membrane, when used according to the invention in Example 8, provides compressor power savings of 11.5% compared to the common membrane from Comparative Example 7, effects energy savings between 7% and 17% when varying various other process parameters in Examples 10 to 17, and at the same time results in an increase of the H₂ content in the purified methane to at least 8.5 vol %, which is especially desirable in the future.

### Example 18, Comparative Example 16

Finally, the process parameters selected for the comparison of membranes in Example 12 and Comparative Example 10 were used again in order to compare the same membrane (see also Comparative Examples 4 and 7) having a selectivity ratio α1/α2 of 80/45=1.8 as well as the one of Comparative Examples 3 and 6 having a selectivity ratio α1/α1 of 190/70=2.7 to the membrane of Example 7.

The latter is, according to Ahmadpour et al. (see above), a PVD/PEBA composite membrane and has a selectivity ratio α1/α2 of 2.5/35=0.07 and thus the lowest ratio found in the literature for separations of H₂ or CO₂, respectively, from CH₄.

In addition, no fixed upper limits for the H₂ content in the purified methane were preset in these comparisons.

The results are summarized in Table 5 overleaf.

TABLE 5

| | Membrane | | Methanation product gas | | | | Permeate | | | Retentate | | Compressor | |
|---|---|---|---|---|---|---|---|---|---|---|---|---|---|
| | selectivity | | | | CO₂ | H₂ | | CO₂ | H₂ | CO₂ | H₂ | req. | |
| | H₂/CH₄ | CO₂/CH₄ | Vol. flow | Pressure | content | content | Pressure | content | content | content | content | power | Saved |
| Example | (α1) | (α2) | [Sm³/h] | [bar] | [vol %] | [vol %] | [bar] | [vol %] | [vol %] | [vol %] | [vol %] | [kW] | energy |
| B18 | 2.5 | 35 | 6000.0 | 30.0 | 3.0 | 12.0 | 2.0 | 14.8 | 19.2 | 0.5 | 10.5 | 277 | 27% |
| V10 | 80 | 45 | | | | | | 11.0 | 47.5 | 0.5 | 0.9 | 379 | |
| B18 | 2.5 | 35 | 6000.0 | 30.0 | 3.0 | 12.0 | 2.0 | 14.8 | 19.2 | 0.5 | 10.5 | 277 | 23% |
| V16 | 190 | 70 | | | | | | 11.5 | 51.1 | 0.5 | 0.5 | 360 | |

It is obvious that energy savings due to the reduced required compressor power were much higher in this case than in Table 4 above in case of the inventive use of the membrane having a selectivity ratio α1/α2 of 2.5/26=0.1, namely another 50% higher than before.

This entailed an H₂ content in the purified methane of 10.5 vol %, however, it is obvious that the results would not have been any worse if it had been limited to 10.0 vol %.

For a person ordinarily skilled in the art it follows that with the development of polymer membranes, such as elastomer membranes, with even lower selectivity ratios α1/α2, the present invention will most likely allow even higher energy efficiency when purifying the product gases of methanations.

In any case, the inventor is at the moment conducting further research and experiments to determine other gas separation membranes suitable according to the present invention in analogy to the ones described above.

The present invention thus provides a new method for producing methane by methanation and subsequent purification via gas membrane separation, which method is not only, but mainly extremely advantageous compared to the method of the state of the art when very low limit values for the concentration of CO₂ in the purified methane have to be complied with.

1. A polymer separation membrane for selectively separating CO₂ and H₂ from CH₄ in a membrane separation step for purifying methane contained in an optionally pre-dried product gas mixture of a methanation method which comprises CH₄, H₂ and CO₂,
wherein
a) the separation is carried out at an operation temperature T_{B} between −20° C. and 100° C.; and
b) the polymer membrane
b1) is able to simultaneously separate CO₂ and H₂ from CH₄,
b2) has a higher selectivity for the separation of CO₂ than of H₂ from CH₄, i.e., a ratio α1/α2<1, and
b3) has a glass transition temperature T_{g} that is lower than the operation temperature T_{B}.

2. A method for producing methane, comprising the following steps:
a methanation step in which, by reducing CO₂ with H₂, a product gas is formed that comprises H₂O, H₂ and CO₂ in addition to CH₄;
optionally a drying step in which H₂O is removed from the product gas; and
a membrane separation step for purifying the methane, wherein the gas mixture obtained by drying and containing CH₄, H₂ and CO₂ is subjected to separation using separation membranes being able to selectively separate CO₂ and H₂ from CH₄;
wherein
a) the separation in the membrane separation step is conducted at an operation temperature T_{B} between −20° C. and 100° C.; and
b) polymer membranes are used that
b1) are able to simultaneously separate CO₂ and H₂ from CH₄,
b2) have a higher selectivity for the separation of CO₂ than of H₂ from CH₄, i.e., a ratio α1/α2<1, and
b3) have a glass transition temperature T_{g} that is lower than the operation temperature T_{B}.

3. The method according to claim 2, wherein in the membrane separation step,
the content of CO₂ in the purified methane is lowered to below 2 vol %, below 1 vol % or below 0.5 vol %; and/or
the content of H₂ in the purified methane is lowered to below 10 vol %, below 8 vol %, below 4 vol %, or below 2 vol %.

4. The method according to claim 2, wherein the separation membranes used are those made of polyethers, poly(urethane-urea) elastomers, polyethers, polysiloxanes, and thermoplastic polyether-blockpolyamide (PEBA) copolymers.

5. The method according to claim 3, wherein the separation membranes used are PEBA copolymer membranes.

6. The method according to claim 2, wherein the separation is conducted at an operation temperature T_{B} between 0° C. and 60° C.

7. The method according to claim 6, wherein the separation is conducted at an operation temperature T_{B} between 5° C. and 30° C.

8. The method according to claim 7, wherein the separation is conducted at an operation temperature T_{B} between 10° C. and 25° C.

9. The method according to claim 2, wherein the separation membranes are able to, in addition to CO₂ and H₂, simultaneously also separate residual amounts of H₂O from CH₄.

10. The polymer separation membrane according to claim 1, wherein in the membrane separation step,
the content of CO₂ in the purified methane is lowered to below 2 vol %, below 1 vol % or below 0.5 vol %; and/or
the content of H₂ in the purified methane is lowered to below 10 vol %, below 8 vol %, below 4 vol %, or below 2 vol %.

11. The polymer separation membrane according to claim 1, wherein the separation membrane comprises a polyether, a poly(urethane-urea) elastomer, a polyether, a polysiloxane, and/or a thermoplastic polyether-block-polyamide (PEBA) copolymer.

12. The polymer separation membrane according to claim 10, wherein the separation membrane comprises a PEBA copolymer membrane.

13. The polymer separation membrane according to claim 1, wherein the separation is conducted at an operation temperature T_{B} between 0° C. and 60° C.

14. The polymer separation membrane according to claim 13, wherein the separation is conducted at an operation temperature T_{B} between 5° C. and 30° C.

15. The polymer separation membrane according to claim 14, wherein the separation is conducted at an operation temperature T_{B} between 10° C. and 25° C.

16. The polymer separation membrane according to claim 1, wherein the separation membrane is able to, in addition to CO₂ and H₂, simultaneously also separate residual amounts of H₂O from CH₄.
